SEQUENCE LISTING (4 sequences)

SEQ ID NO: 1 - RNA, 21 nt, synthetic construct
  source 1..21: mol_type = other RNA
  modified_base 20..21: mod_base = OTHER; note = thymine
gggacatcaatcgtatttttt

SEQ ID NO: 2 - RNA, 21 nt, synthetic construct
  source 1..21: mol_type = other RNA
  modified_base 20..21: mod_base = OTHER; note = Thymine
aaaatacgattgatgtccctt

SEQ ID NO: 3 - DNA, 21 bp, synthetic construct
  source 1..21: mol_type = other DNA
gtcccatgaacctcgatttaa

SEQ ID NO: 4 - DNA, 21 bp, synthetic construct
  source 1..21: mol_type = other DNA
gtgaatccagatagttgtatc

(19) United States
(12) Patent Application Publication
(10) Pub. No.: US 2026/0158032 A1
(43) Pub. Date: Jun. 11, 2026

(54) PARP AND ADAR1 INHIBITION FOR CANCER TREATMENT

(71) Applicant: UNIVERSITY OF VIRGINIA PATENT FOUNDATION, Charlottesville, VA (US)

(72) Inventors: Qiwei Wang, Charlottesville, VA (US); Sophie O’Keefe, Charlottesville, VA (US)

(21) Appl. No.: 19/401,202

(22) Filed: Nov. 25, 2025

Related U.S. Application Data
(60) Provisional application No. 63/724,748, filed on Nov. 25, 2024.

(51) Int. Cl. A61K 31/502; A61K 31/496; A61K 31/5025; A61K 31/7105; A61K 39/395; A61P 35/00

(52) U.S. Cl. CPC A61K 31/502; A61K 31/496; A61K 31/5025; A61K 31/7105; A61K 39/3955; A61P 35/00

(57) ABSTRACT

Methods for treating a cancer comprising administering an ADAR1 inhibitor and a PARP inhibitor are described herein. PARP inhibition enhances tumor sensitivity to ADAR1 inhibition, leading to enhanced cancer cell death. The described combination therapy is effective for treating breast cancers characterized by a loss of p53 and BRCA1 or PTEN, which are typically resistant to PARP inhibition therapy alone.

### CROSS-REFERENCE TO RELATED APPLICATION(S)

This application claims the benefit of and priority to U.S. Provisional Application No. 63/724,748 filed on Nov. 25, 2024, the content of which is incorporated by reference in its entirety.

### STATEMENT REGARDING FEDERALLY SPONSORED RESEARCH

This invention was made with government support under GR103766 awarded by the Department of Defense. The government has certain rights in the invention.

### REFERENCE TO AN ELECTRONIC SEQUENCE LISTING

The content of the electronic sequence listing (18250600060.xml; Size: 5,443 bytes; and Date of Creation: Nov. 25, 2025) is herein incorporated by reference in its entirety BACKGROUND

Breast and prostate cancer are the first and second most common cancers in the United States. Pancreatic cancer accounts for 8% of all annual cancer deaths, and ovarian cancer has a 50.9% 5-year survival rate. Previous studies have demonstrated that cancers with elevated Interferon Signaling Genes (ISGs) have an increased dependency on ADAR1. However, most cancer cells intrinsically downregulate the expression of ISGs to evade immune surveillance. Strategies for pharmacologically eliciting tumor-specific ADAR1 dependency are of interest.

### SUMMARY

In an aspect, provided herein is a method of treating a cancer in a subject in need thereof, the method comprising: administering to the subject a first pharmaceutical composition comprising an effective amount of an ADAR1 inhibitor and a pharmaceutically acceptable carrier; and administering to the subject a second composition comprising an effective amount of a PARP inhibitor and a pharmaceutically acceptable carrier.

The PARP inhibitor may be selected from olaparib, talazoparib, and saruparib. The ADAR1 inhibitor may comprise a gene silencing agent or a small molecule compound. The gene silencing agent may be in a delivery vehicle. The delivery vehicle may be a lentiviral vector or a nanocarrier. The gene silencing agent may be selected from an siRNA, an shRNA, an miRNA, and an antisense oligonucleotide. The siRNA may comprise a sense strand comprising SEQ ID NO: 1, and an antisense strand comprising SEQ ID NO: 2. In embodiments, the ADAR1 inhibitor is administered before the PARP inhibitor.

The method may further comprise administering to the subject an immunotherapy agent. The immunotherapy agent may comprise an anti-PD-L1 antibody or an anti-PD-1 antibody.

The subject may have a breast cancer or an ovarian cancer. The subject may have a cancer characterized by a loss of BRCA1 and p53. The subject may have a cancer characterized by the loss of PTEN and p53. The subject may be a human.

### BRIEF DESCRIPTION OF THE DRAWINGS

FIGS. 1A-1. BP tumor cells treated with or without olaparib (10 μM, 1 day) were subjected to RNA sequencing (RNA-seq). (A) Gene set enrichment analysis (GSEA) of BP+olaparib compared to control BP cells. Normalized Enrichment Score, NES. (B) Gene expression measured by FPKM (short for the expected number of Fragments Per Kilobase of transcript sequence per Millions base pairs sequenced), a common method of estimating gene expression levels, which takes the effects into consideration of both sequencing depth and gene length on counting of fragments.

FIGS. 2A-2F. (A-C) BP and PP tumor cells were transfected with Adar1 siRNA (20 μM) or control siRNA and treated with or without olaparib (OL, 10 μM for 2 days). Cells were then stained with Annexin V and 7AAD followed by flow cytometry analysis. (A) Representative Western blots of ADAR1 and β-Actin. (B) Representative plots of flow cytometry. (C) BP and PP cells were analyzed for apoptosis (Annexin V+ 7-AAD−). (D-F) BP and PP tumor cells were lentivirally transduced with Adar1 shRNA to achieve stable knockdown of Adar1. Two different Adar1 shRNA sequences were used (1 and 2). shGFP was used as a control. Cells were then treated with or without olaparib for 2 days followed by Annexin V/7AAD staining and flow cytometry. (D) Representative Western blots of ADAR1 and β-Actin. (E and F) BP and PP cells were analyzed for apoptosis (Annexin V+7-AAD−). n=4 (C) or 3 (E and F). One-way ANOVA. *P<0.05, **P<0.005, ***P<0.0005, ****P<0.0001. ns, not significant.

FIGS. 3A-3E. BP tumor cells were transfected with Adar1 siRNA (20 μM) or control siRNA and treated with or without olaparib (OL, 10 μM for 2 days). Cells were then subjected to RNA sequencing (RNA-seq). (A) A heat map of differentially expressed genes. (B) Gene set enrichment analysis (GSEA) of Gene Ontology (GO) and Kyoto Encyclopedia of Genes and Genomes (KEGG) comparing BP cells+Adar1 siRNA+OL with BP cells+Control siRNA+OL. (C-E) Gene expression measured by FPKM (short for the expected number of Fragments Per Kilobase of transcript sequence per Millions base pairs sequenced), a common method of estimating gene expression levels, which takes the effects into consideration of both sequencing depth and gene length on counting of fragments.

FIGS. 4A-4B. (A) PP-shAdar1 cells were treated with or without olaparib (OL, 10 μM for 2 days) in presence or absence of Ruxolitinib (1p M). Cells were then stained with Annexin V and 7AAD, followed by flow cytometry analysis of apoptosis (Annexin V+7AAD−). n=3. One-way ANOVA. ****P<0.0001. (B) Proposed mechanisms underlying tumor cell lethality induced by combined PARP inhibition and ADAR1 inhibition.

FIG. 5. Brca1^{-/-}Trp53^{-/-} murine TNBC cells were subjected to transcriptome analysis after being treated for 48 h with DMSO vehicle control or olaparib (10 μM) for 2 days. Volcano plot showing the significance and magnitude of changes in gene expression in olaparib-treated cells compared to the DMSO vehicle control group. Genes that are significantly upregulated in the olaparib-treated group and related to dsRNA are highlighted.

FIG. 6. PARP inhibition induced accumulation of cytoplasmic dsRNA. Top, representative immunofluorescence images of dsRNA (red) staining in Pten^{-/-}Trp53^{-/-} murine TNBC cells treated with or without 10 μM Olaparib for 3 days. Nuclei were counterstained with DAPI. Scale bar, 50 μm. Bottom, quantification of cytoplasmic dsRNA in Pten^{-/-}Trp53^{-/-} murine TNBC cells and in human breast cancer cell lines BT549 and MCF7 treated with or without 10 μM Olaparib for 3 days.

FIG. 7. Quantitative PCR (qPCR) results of transposable elements in Pten^{-/-}Trp53^{-/-} murine TNBC cells treated with olaparib (5 μM, 72 hours) or DMSO vehicle control.

FIG. 8. Pten^{-/-}Trp53^{-/-}, Brca1^{-/-}Trp53^{-/-}, and 4T1 murine TNBC cells and human TNBC cell line BT549 were treated with control or ADAR1 siRNA (20 nM) in the presence of olaparib (5 μM) or DMSO vehicle control for 2 days, followed by Annexin V and 7-AAD staining. Cells were analyzed by flow cytometry for apoptosis (Annexin V+7-AAD−) (n=2-5). Data are presented as mean SEM. One-way ANOVA. *p<0.05, **p<0.01, ***p<0.001, ****p<0.0001

FIG. 9. Pten^{-/-}Trp53^{-/-}-shGFP (Top) and Pten^{-/-}Trp53^{-/-}-shAdar1 (Bottom) tumor growth in FVB mice treated with olaparib (50 mg/kg, IP, QD). Treatment was initiated when mammary tumors became palpable. The x-axis indicates the number of days of treatment. n=9 tumors (Pten^{-/-}Trp53^{-/-}-shGFP); 8 tumors (Pten^{-/-}Trp53^{-/-}-shGFP); 6 tumors (Pten^{-/-}Trp53^{-/-}-shAdar1); 10 tumors (Pten^{-/-}Trp53^{-/-}-shAdar1+Olaparib). Data are presented as mean±SEM.

### DETAILED DESCRIPTION

The inventors have demonstrated that PARP inhibition elevates interferon signaling in cancers such as breast cancer, regardless of BRCA1/2 status, and renders them dependent upon ADAR1, making them susceptible to pharmacologic inhibition of ADAR1.

In a first aspect, provided herein is a method of treating a cancer in a subject in need thereof, the method comprising: administering to the subject a first pharmaceutical composition comprising an effective amount of an ADAR1 inhibitor and a pharmaceutically acceptable carrier; and administering to the subject a second composition comprising an effective amount of a PARP inhibitor and a pharmaceutically acceptable carrier.

ADAR1 is an enzyme that binds to double-stranded RNA (dsRNA) and converts adenosine to inosine (A to I) by deamination. This A to I conversion disrupts the normal A:U pairing, destabilizing the RNA. ADAR1 has two known isoforms, ADAR1 p150 and ADAR1 p110. The p110 isoform is typically found in the nucleus, while the p150 isoform is found in both the nucleus and the cytoplasm, but is mostly present in the cytoplasm. In embodiments, the ADAR1 inhibitor selectively targets the p150 isoform, and does not target the p110 isoform. The ADAR1 inhibitor may comprise a gene silencing agent or a small molecule compound.

A gene silencing agent may reduce expression of a target gene or a molecule that positively regulates said target gene. The gene silencing agent may comprise a nucleic acid sequence that is capable of inducing RNA interference (RNAi). The term “RNA interference” refers to a process in which RNA molecules inhibit gene expression or translation by neutralizing targeted mRNA molecules. To achieve an RNAi effect, for example, RNA having a double strand structure containing the same base sequence as that of the target mRNA may be used. Gene silencing agents include, but are not limited to, small interfering RNA (siRNA), short hairpin RNA (shRNA) microRNA (miRNA), miRNA zippers, and antisense oligonucleotides (ASOs). Any RNA molecule that is capable of silencing gene expression of a target gene may be used in connection with the present disclosure.

The term “delivery vehicle”, as used herein, refers to a molecular compound suitable for delivering the gene silencing agent to a cell. Delivery vehicles include, but are not limited to, vectors, lipid nanoparticles, and polymeric nanoparticles. In embodiments, the gene silencing agent is in a construct and operably linked to a promoter. The term “construct” refers to a recombinant polynucleotide, i.e., a polynucleotide that was formed artificially by combining at least two polynucleotide components from different sources (natural or synthetic). For example, the construct may comprise promoters and elements that are (1) associated with another gene found within the same genome, (2) from the genome of a different species, or (3) synthetic. As used herein, the term “promoter” refers to a DNA sequence that regulates the transcription of a polynucleotide. Typically, a promoter is a regulatory region that is capable of binding RNA polymerase and initiating transcription of a downstream sequence. Promoters may be derived in their entirety from a native gene, may be composed of elements derived from multiple regulatory sequences found in nature, or may comprise synthetic DNA segments. A promoter is “operably linked” to a polynucleotide if the promoter is connected to the polynucleotide such that it may affect transcription of the polynucleotide. Constructs can be generated using conventional recombinant DNA methods. In embodiments, the construct is packaged in a vector. As used herein, the term “vector” refers to a virus particle that is used to deliver genetic material into cells. Vectors include, without limitation, a yeast artificial chromosome, bacterial plasmid (e.g., naked or contained in liposomes), phagemid, shuttle vector, cosmid, virus (e.g., Sendai viruses, lentiviruses, retroviruses, adenoviruses, and adeno-associated viruses), chromosome, mitochondrial DNA, plasmid DNA, and nucleic acid fragment. In exemplary embodiments, the gene silencing agent is an shRNA packaged in a lentiviral vector.

The term “nanocarrier,” “nanoparticle,” or “nanostructure” refers to a nanomaterial used as a transport module for another substance. Nanocarriers include, without limitation, polymeric nanoparticles, carbon-based materials, dendrimers, and lipid-based carriers, such as liposomes. For example, the gene silencing agent may be an siRNA in a lipid nanocarrier.

In embodiments, the ADAR1 gene silencing agent is an siRNA. The siRNA may comprise a sense strand comprising SEQ ID NO: 1 or a sequence having at least 90% identity thereto, and an antisense strand comprising SEQ ID NO: 2 or a sequence having at least 90% identity thereto. In embodiments, the ADAR1 gene silencing agent is an shRNA.

As used herein, the terms “small molecular therapeutic agent”, “small molecule compound”, and “small molecule drug” refer to a chemical compound or pharmaceutically acceptable salt thereof having a therapeutic effect and/or enhancing the therapeutic effect of an immunotherapy. Small-molecule drugs are typically comprised of 20 to 100 atoms and have a molecular mass of less than 1000 g/mol or 1 kilodalton [kDa]. Small-molecules drugs can typically be administered by a variety of routes (including orally) and can pass through cell membranes to reach intercellular targets.

Suitable small molecule ADAR1 inhibitors include, but are not limited to,

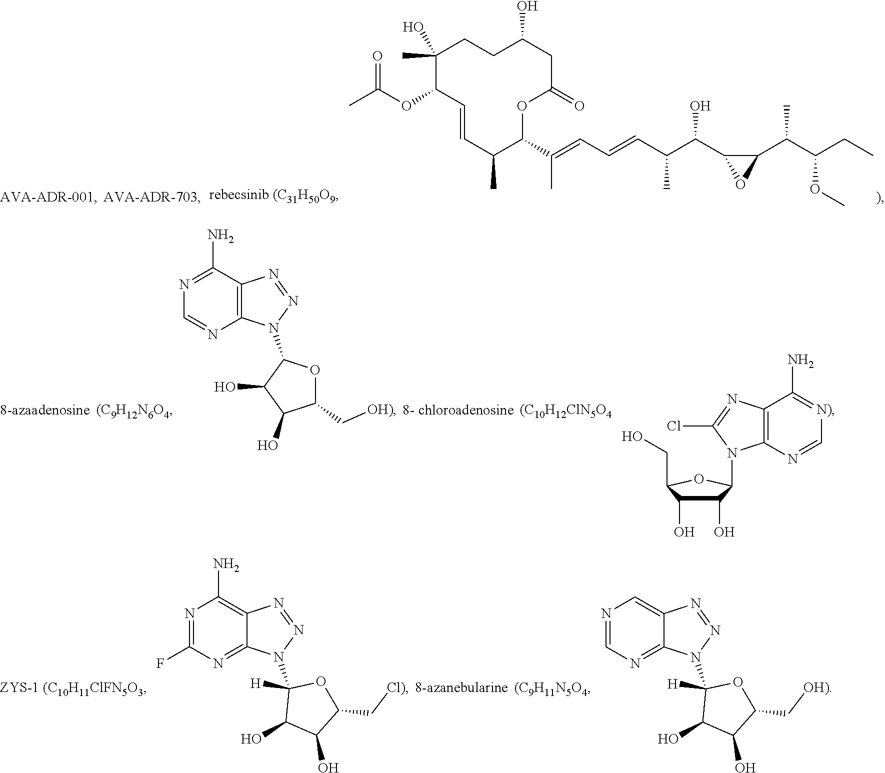

PARP is a protein involved in cellular processes including DNA repair, genomic stability, and programmed cell death. Its main role is to detect and initiate an immediate cellular response to metabolic, chemical, or radiation-induced single-strand DNA breaks (SSB) by signaling the enzymatic machinery involved in the SSB repair. The PARP inhibitor may comprise a gene silencing agent or a small molecule compound. The small molecule PARP inhibitor may be selected from olaparib, talazoparib, saruparib, rucaparib, veliparib, and niraparib. In exemplary embodiments, the PARP inhibitor is olaparib.

In exemplary embodiments, the ADAR1 inhibitor and the PARP inhibitor are administered orally. In embodiments, the ADAR1 inhibitor is administered at a dose of between about 30 and about 60 mg. In embodiments, the ADAR1 inhibitor is administered daily. In embodiments the PARP inhibitor is Olaparib. The Olaparib may be administered at about 150 mg (capsule) daily or about 100 mg (tablet) twice a day. In embodiments the PARP inhibitor is talazoparib. The talazoparib may be administered at between about 0.5 and about 0.75 mg daily. In embodiments, the ADAR1 inhibitor is administered at the same time as the PARP inhibitor. For example, the ADAR1 inhibitor and the PARP inhibitor may be formulated together in a composition, and the composition may be administered. In embodiments, the ADAR1 inhibitor is administered after administration of the PARP inhibitor. In embodiments, the ADAR1 inhibitor is administered after a 1-2 week treatment with the PARP inhibitor. In embodiments, the ADAR1 inhibitor is administered before administration of the PARP inhibitor. In embodiments, the ADAR1 inhibitor is administered before a 1-2 week treatment with the PARP inhibitor.

The method may further comprise administering to the subject an effective amount of an immunotherapy agent. The term “immunotherapy” refers to treatment of disease by activating or suppressing the immune system. For example, immunotherapy as applied to a cancer relevant to the present application may include the use of agents that target checkpoint inhibition (e.g., immune checkpoint inhibitors). For instance, the immunotherapy may include agents (e.g., antibodies (neutralizing antibodies), small-molecule inhibitors, protein-binding peptides) that bind and neutralize the action of immune checkpoint proteins that regulate the immune response. The neutralization of immune checkpoint proteins can lead to increased activity of T cells, resulting in increased targeting and killing of cancer cells. Immunotherapy agents include any type of antibody or antibody-like peptides including but not limited to nanobodies, fragmented antibodies or fragmented antibody derivatives (e.g., Fab, Fab′, F(ab′)2, sdAb, scFv, di-scFv), peptide/MHC-complexes, cell adhesion receptor molecules, receptors for costimulatory molecules, and artificial engineered binding molecules (e.g., aptamers). In embodiments, the immune checkpoint inhibitors target one or more proteins involved in the immune checkpoint pathway. The one or more targeted immune checkpoint proteins may be selected from PD-1 and PD-L1. For example, the immunotherapy may include the administration of an antibody or small molecular compound against PD-1 and/or PD-L1 to a subject for the treatment of cancer. The anti-PD-L1 therapy may be an anti-PD-L1 antibody. The anti-PD-1 therapy may be an anti-PD-1 antibody.

The ADAR1 inhibitor, PARP inhibitor, anti-programmed death-ligand 1 (PD-L1) therapy or anti-programmed cell death protein 1 (anti-PD-1) therapy, and any additional therapeutic agent described herein may be administered in any order. Any of the ADAR1 inhibitor, PARP inhibitor, anti-PD-L1 therapy or anti-PD-1 therapy, and any additional therapeutic agent described herein may be formulated as a pharmaceutical composition. The composition may comprise the agent(s) and a pharmaceutically acceptable carrier.

The method may further comprise administering to the subject an additional therapeutic agent that activates interferon signaling. The therapeutic agent may comprise a Stimulator of Interferon Genes (STING) agonist or a chemotherapy. STING agonists include, but are not limited to,

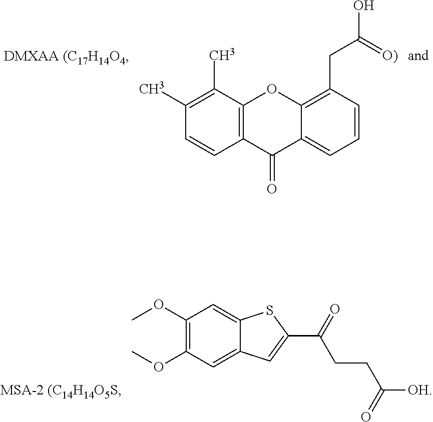

Inert ingredients and manner of formulation of the pharmaceutical compositions are conventional. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active chemical compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Capsules are prepared by mixing the chemical compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders. Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The chemical compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects. A lubricant can be used in a tablet formulation to prevent the tablet and punches from sticking in the die. The lubricant can be chosen from such slippery solids as talc, magnesium and calcium stearate, stearic acid, and hydrogenated vegetable oils. Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The cancer may be any cancer including, but not limited to, a breast cancer, a prostate cancer, an ovarian cancer, a pancreatic cancer. The cancer may comprise a BRCA1 mutation, a BRCA2 mutation, or both. Breast cancer type 1 susceptibility protein (BRCA1) is encoded by the BRCA1 tumor suppressor gene. Breast cancer type 2 susceptibility protein (BRCA2) is encoded by the BRCA2 tumor suppressor genes. Hundreds of different BRCA1 and BRCA2 mutations have been identified, some which increase the risk of occurrence of breast cancer and/or ovarian cancer.

The cancer may comprise a mutation in p53, also known as cellular tumor antigen p53 or transformation-related protein 53 (TRP53). The p53 protein is a regulatory transcription factor often mutated in human cancers.

The cancer may comprise a phosphatase and tensin homolog (PTEN) mutation. PTEN is a tumor suppressor gene, and mutations are linked to many cancers, including breast cancer.

As used herein, the term “administering”, refers to dispensing, delivering, or applying the therapeutic agent (e.g. ADAR1 inhibitor, PARP inhibitor, etc.), to a subject by any suitable route for delivery of the substance to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route. The preferred route may vary with, for example, the subject's pathological condition or age or the subject's response to therapy or that is appropriate to the circumstances. The formulations can also be administered by two or more routes, where the delivery methods are essentially simultaneous, or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

The terms “treating” and “to treat” includes the reducing, repressing, delaying or preventing cancer growth and/or metastasis.

The terms “effective amount” or “therapeutically effective amount” refer to an amount sufficient to effect beneficial or desirable biological and/or clinical results. The amount of the pharmaceutical composition that is therapeutically effective may vary depending on the particular pathogen or the condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized.

The terms “subject” and “patient” are used herein interchangeably to refer to a mammal, preferably a human, to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Preferably, the subject is a human.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The cited references are incorporated by reference herein in their entireties. In the event that there is an inconsistency between a definition of a term in the specification as compared to a definition of the term in a cited reference, the term should be interpreted based on the definition in the specification.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLE

### Background

Breast and prostate cancer are the first and second most common cancers in the United States (1). Pancreatic cancer accounts for 8% of all annual cancer deaths, and ovarian cancer has a 50.9% 5-year survival rate (2). Despite the heterogeneity of these cancers, breast, prostate, pancreatic, and ovarian cancer are all approved for PARP inhibitor (PARPi) therapy when the patient has a pathogenic mutation in the BRCA1/2 genes (3).

PARP inhibitors show variable efficacy across different cancer types. For example, PARPi significantly increased overall survival (OS) in patients with advanced ovarian cancer but not in patients with advanced breast cancer (4-7). Of note, OS is not only a direct measure of clinical benefit but also a patient-centered measure. Currently, there are no approved treatment strategies that can effectively and predictably enhance a patient's response to PARPi across cancer types.

PARPi were initially designed to exploit synthetic lethality in cancers with homologous recombination (HR) deficiencies, such as those characterized by BRCA1/2 pathogenic mutations (8). However, recent evidence, including our own work and that of others, reveals that the immunomodulatory function of PARPi is equally crucial for their therapeutic efficacy. Mechanistically, PARPi stimulates a robust antitumor immune response, both locally and systemically, engaging both innate and adaptive immunity. This response is mediated by the STING pathway, leading to the activation of type I interferon signaling (9). Therefore, the full therapeutic potential of PARPi relies on the synergistic effects of both synthetic lethality and immune activation. However, the full therapeutic potential of PARPi has yet to be fully explored.

There are two major ongoing efforts in the clinic: (1) enhancing PARPi efficacy in BRCA-associated cancers through rational combinations; (2) expanding the use of PARPi to extend the benefits beyond BRCA-associated cancers.

### Targeting ADAR1 (Adenosine Deaminase Acting on RNA 1) for Cancer Therapy

The RNA editing protein ADAR1 is one of the most high-value oncology targets. ADAR1 mediates A-to-I RNA editing, which is a key post-transcriptional modification that converts adenosines to inosines in both coding and noncoding RNA transcripts. In cancer cells, ADAR1 deficiency increases the amount of unedited double-stranded RNA (dsRNA), leading to activation of dsRNA sensors, such as melanoma differentiation-associated gene 5 (MDA5), protein kinase R (PKR), oligoadenylate synthases, and Z-DNA/RNA binding protein 1 (ZBP1). The activation of these proteins triggers interferon (IFN) production and cell death (10).

However, ADAR1 inhibition as a monotherapy failed to invariably provide antitumor efficacy in solid tumors (11,12). Current studies mainly focus on combining ADAR1 inhibitors with immune checkpoint blockade (e.g., anti-PD1 and anti-PD-L1) (13). Still, the response to this combination is likely to be limited by an immunosuppressive tumor microenvironment (TME).

Previous studies have demonstrated that cancers with elevated Interferon Signaling Genes (ISGs) have an increased dependency on ADAR1 (12). However, most cancer cells intrinsically downregulate the expression of ISGs to evade immune surveillance. It remains unclear how to pharmacologically elicit tumor-specific ADAR1 dependency. We hypothesized that PARP inhibition will elevate ISGs regardless of BRCA1/2 status, and render cancer cells dependent upon ADAR1, making them susceptible to pharmacologic inhibition of ADAR1.

### Results

### PARPi Treatment Increases Expression of ISGs, EIF2AK2 (Encodes PKR), and Adar1 in Cancer Cells

Given the high prevalence of BRCA1, P53, and PTEN deficiencies in breast cancer, we previously developed genetically engineered mouse (GEM) models of breast cancer driven by the concurrent loss of BRCA1 and P53 (termed the BP model) or the concurrent loss of PTEN and P53 (termed the PP model). Both BP and PP models are developed using immunocompetent FVB/N mice and therefore are suitable for immuno-oncology studies. Notably, both models are highly aggressive, with BP and PP tumors histologically identified as poorly differentiated adenocarcinomas and triple-negative breast cancer (TNBC), representing advanced and hard-to-treat breast cancer in the clinic (14,15). Indeed, both BP and PP tumors are resistant to currently approved breast cancer therapies, such as PARP inhibitors (a targeted therapy inhibiting DNA damage repair approved for BRCA1-deficient breast cancer) and anti-PD-1 antibody (an immunotherapy approved for TNBC) (14,15).

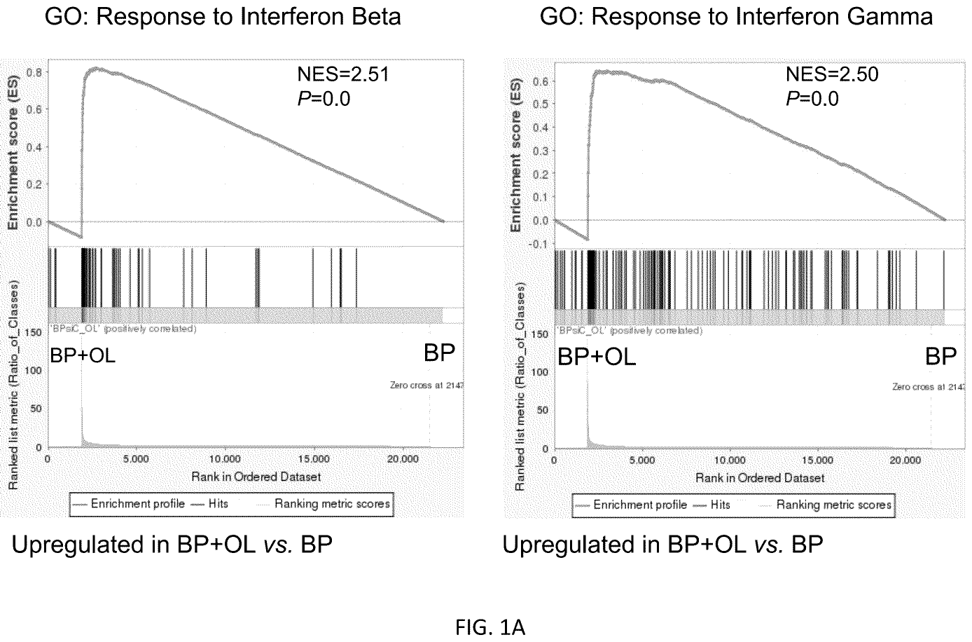

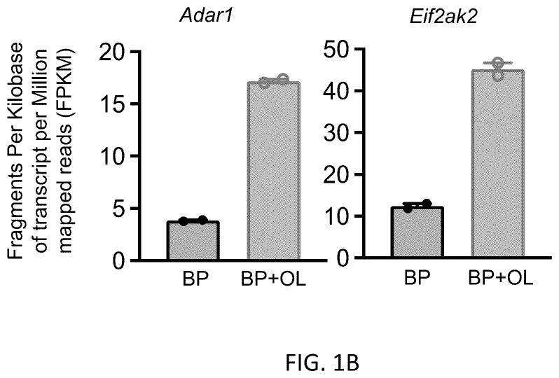

We have previously demonstrated that the BP GEM model of breast cancer shows only a modest response to olaparib, an FDA-approved PAPRi for patients with BRCA1-deficient breast cancer (16). To further investigate the response of BP tumors to PARPi, we treated BP tumor cells with olaparib (10 μM, 2 days) and analyzed the cells by RNA sequencing (RNA-seq). Notably, gene set enrichment analysis (GSEA) of Gene Ontology (GO) suggests that signaling pathways associated with “response to interferon beta” and “response to interferon gamma” are significantly upregulated in olaparib-treated BP tumor cells compared to control BP cells (FIG. 1A). Furthermore, the gene expression of Adar1 and EIF2AK2 (encodes PKR) increased by more than 3-4 fold in BP tumor cells upon olaparib treatment (FIG. 1).

The elevation of interferon signaling and PKR has previously been shown to be associated with increased ADAR1 dependency (12,17). The induction of Adar1 indicates an ADAR1-mediated PARP inhibitor resistance mechanism, warranting further investigation. Taken together, our data suggest a potential synergy to target ADAR1 in the context of olaparib treatment.

Combining PARPi with ADAR1 Targeting Synergistically Induces Cancer Cell Lethality, Regardless of BRCA1/2 Status

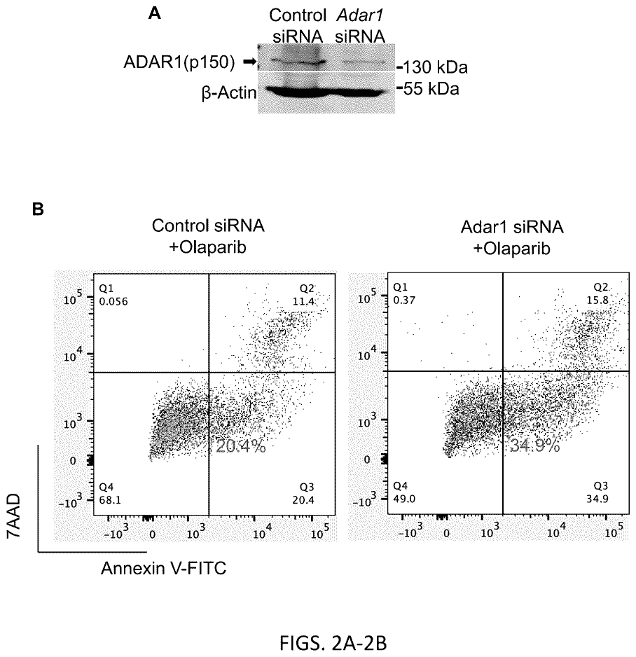

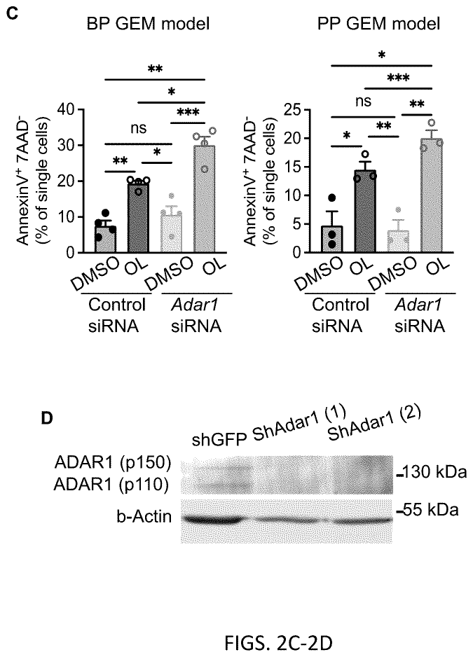

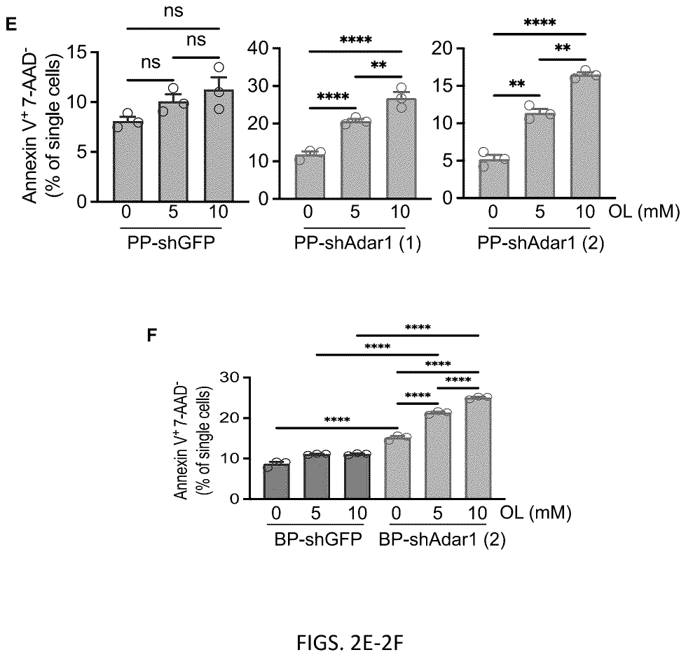

To evaluate the efficacy of combined PARP and ADAR1 inhibition, we first transiently knocked down Adar1 using siRNA (sense strand sense strand: 5′-GGGACAUCAAUCGUAUUUUTT-3′ (SEQ ID NO: 1), antisense strand: 5′-AAAAUACGAUUGAUGUCCCTT-3′ (SEQ ID NO: 2)) and confirmed the reduction of ADAR1 protein by Western blot (FIG. 2A). We then treated these cells with olaparib. As shown in FIGS. 2B and C, the efficacy of olaparib significantly increased in ADAR1-deficient cells. Notably, this effect was observed in both BRCA1-deficient (BP cells) and BRCA1/2-proficient (PP cells) breast cancer cells, suggesting that the increased cell lethality induced by PARP inhibition and ADAR1 knockdown does not rely on BRCA1/2 status. To confirm these findings, we further derived ADAR1 stable knockdown tumor cells via lentivirally transduced shRNA (FIG. 2D). (The ADAR1 shRNA plasmids were purchased from Sigma. PLKO.1-U6-shADAR1-puroR. version 1: product #TRCN0000234067, target sequence: GTCCCATGAACCTCGATTTAA (SEQ ID NO: 3). version 2: product #TRCN0000218395, target sequence: GTGAATCCAGATAGTTGTATC) (SEQ ID NO: 4). While BP and PP cells initially showed increased cell death following ADAR1 knockdown, they both survived chronic ADAR1-deficiency, suggesting adaptive resistance to ADAR1 inhibition. Notably, olaparib showed significantly enhanced efficacy in ADAR1-deficient cells compared to ADAR1-proficient cells in both PP and BP models (FIGS. 2E and F). These data suggest that combining PARP inhibitors with ADAR1 targeting synergistically enhances cancer cell lethality, independent of BRCA1/2 status.

Combining PARPi with ADAR1 Targeting Elicits Tumor Cell-Intrinsic Immune Activation and Enhances Tumor Cell Immunogenicity

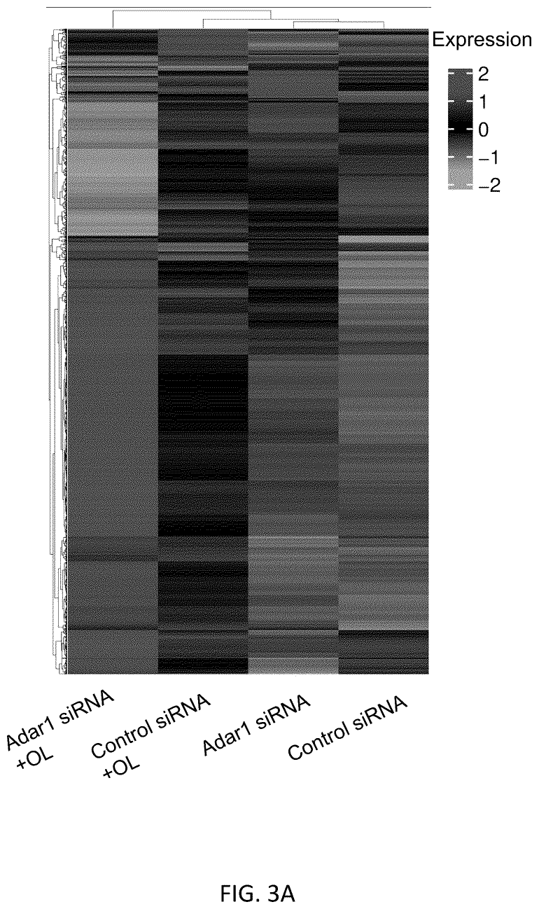

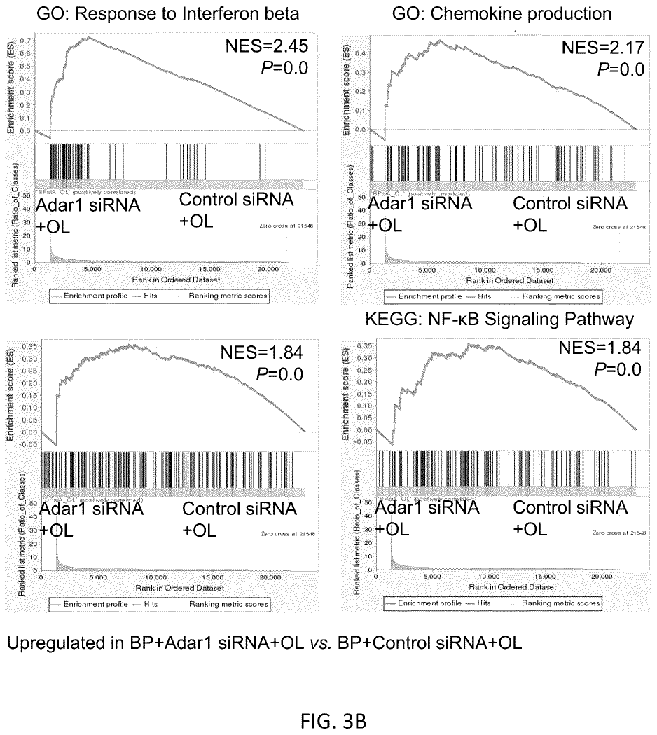

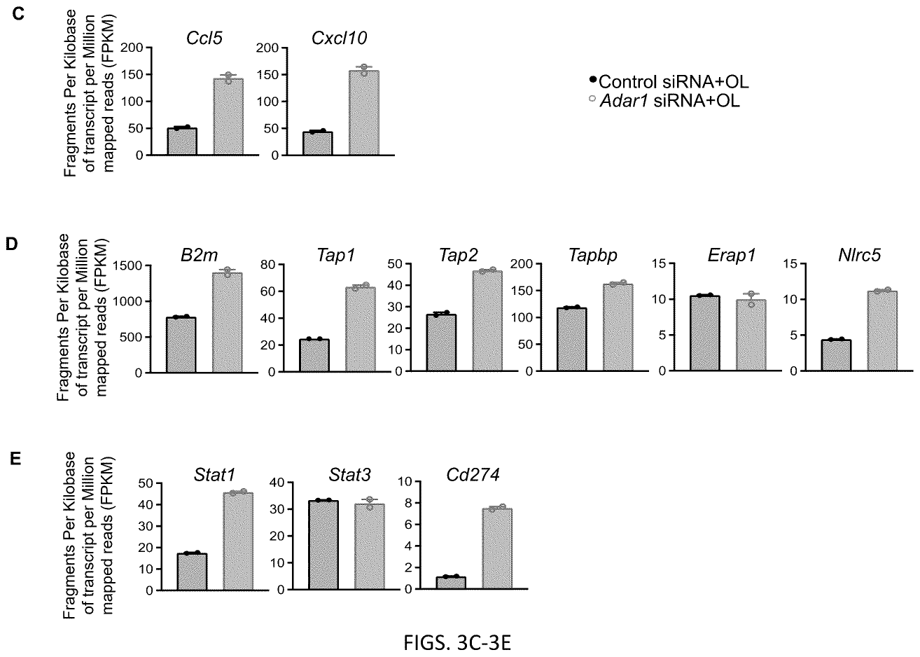

We further investigated the effects of combined PARP inhibition with ADAR1 targeting on tumor cell-intrinsic immunity. BP tumor cells were transiently silenced for ADAR1 expression and treated with olaparib. RNA-seq analysis suggests that tumor cells treated with Adar1 siRNA and olaparib exhibited a distinct transcriptional profile compared to other groups (FIG. 3A). Previous studies have shown that olaparib monotherapy can induce tumor cell-intrinsic immunity as evidenced by increased type I interferon signaling (18). Notably, we found that compared to olaparib monotherapy, combining Adar1 siRNA with olaparib further increased the response to interferon beta (FIG. 3B). Moreover, the combination also increased genes associated chemokine production, JAK-STAT signaling pathway and NF-kB signaling pathway in tumor cells (FIG. 3B). Specifically, BP cells treated with the combination therapy had increased expression of Ccl5 and Cxcl10 (FIG. 3C), which are important for T cell recruitment (19). Treatment with the combination therapy also upregulated genes encoding murine major histocompatibility complex (MHC) class I molecules (B2m), as well as genes involved in peptide transport (Tap1 and Tap2), and transporter-MHC interactions (Tapbp). While Erap1 (which encodes ERAP1, important for peptide cleavage) remained unchanged between the combination treatment and olaparib single agent, Nlrc5, a key regulator of MHC class I-dependent immune responses, increased by more than 2-fold upon the combination treatment (FIG. 3D). Interestingly, the combination treatment upregulated Stat1 expression without increasing Stat3. Notably, the combination therapy led to an approximately 8-fold increase in Cd274 expression in tumor cells. Cd274 encodes PD-L1, a critical immune checkpoint protein. This finding suggests that incorporating anti-PD-L1 therapy could further augment the efficacy of the combination treatment.

Collectively, our data indicate that combining PARP and ADAR1 inhibition enhances both tumor cell-intrinsic immunity and immunogenicity to a greater extent than olaparib monotherapy.

### Targeting JAK1/2 Attenuates the Cell Death Induced by Combined PARP Inhibition and ADAR1 Inhibition

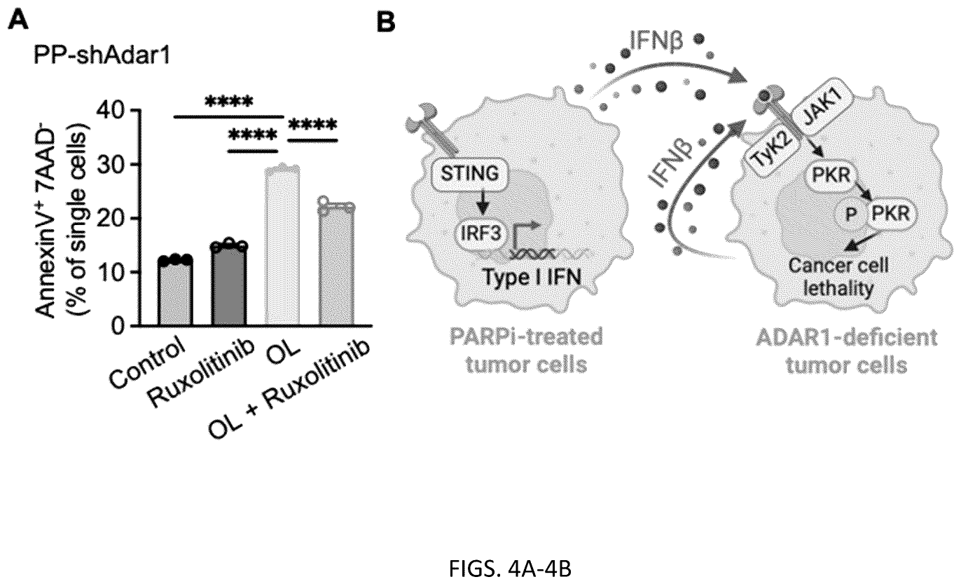

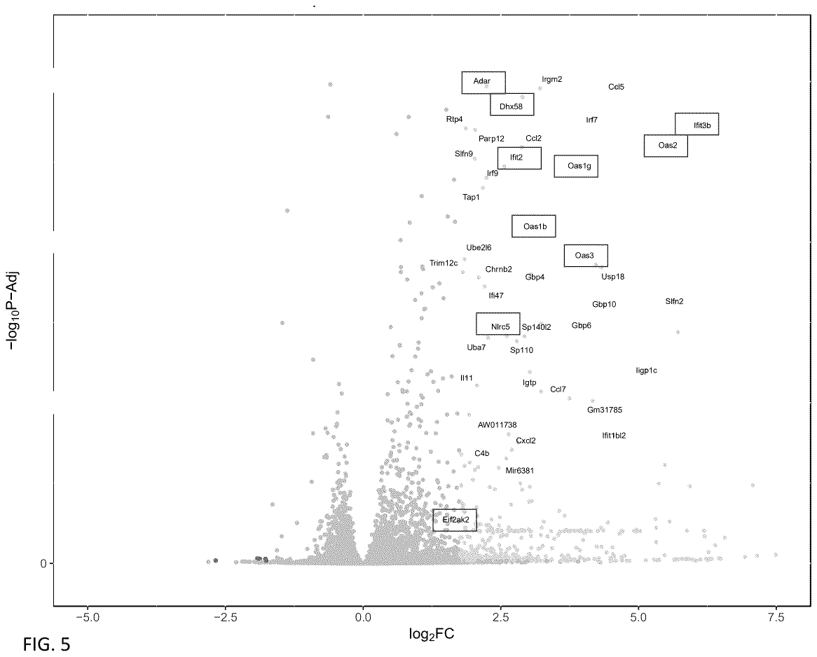

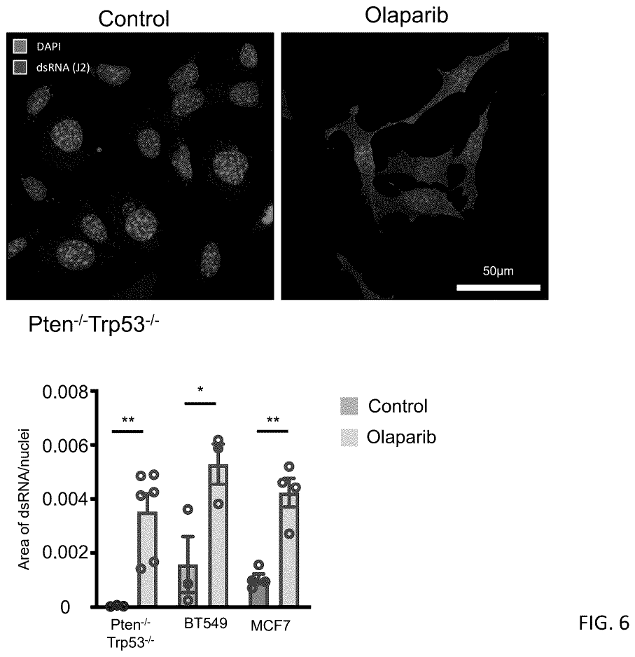

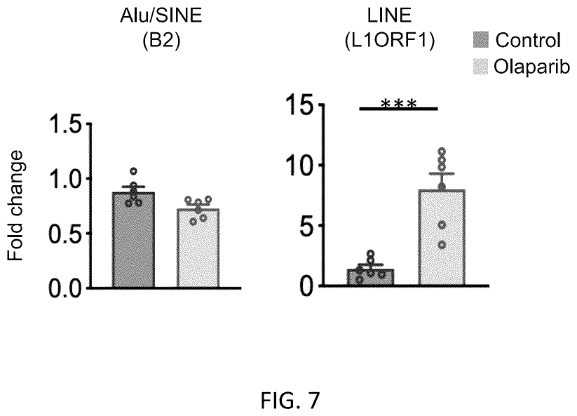

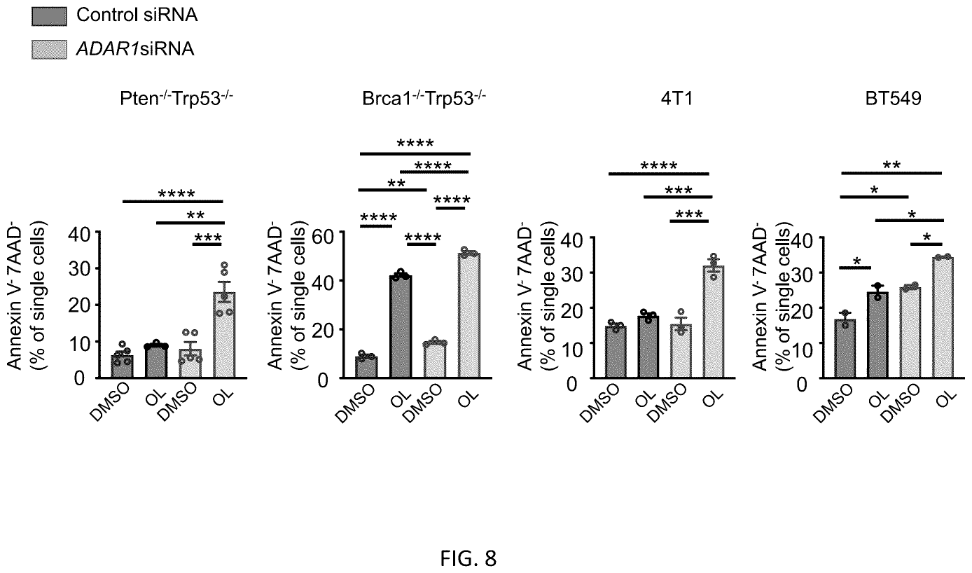

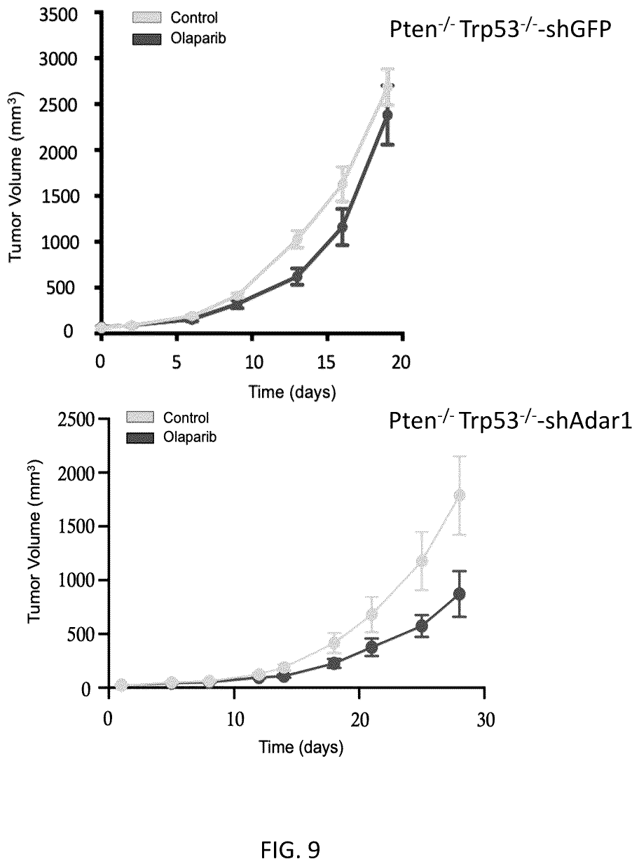

To investigate the mechanism underlying the cell vulnerability, we sought to target cellular response to interferon signaling. JAK1/2 are key downstream targets of interferon signaling (20). We found that inhibiting JAK1/2 with ruxolitinib significantly reduced the cell death triggered by combined PARP and ADAR1 inhibition (FIG. 4A). Thus, as shown in FIG. 4B, we propose a model wherein PARPi treatment triggers intrinsic STING activation within tumor cells, leading to the production of type I interferons. These interferons, released from tumor cells, enhance JAK1/PKR pathway expression via paracrine or autocrine signaling. Elevated levels of the dsRNA sensor PKR sensitize cells to unedited dsRNA. Consequently, ADAR1 inhibition, which causes unedited dsRNA accumulation, synergizes with PARP inhibition, which upregulates PKR expression. This synergy ultimately induces tumor cell death and activates other downstream effectors, such as tumor cell immunogenicity.

### REFERENCES

1. Cancer Stat Facts: Common Cancer Sites. NCI Surveillance, Epidemiology, and End Results Program <https://seer.cancer.gov/statfacts/html/common.html>.
2. Cancer Stat Facts: Ovarian Cancer. NCI Surveillance, Epidemiology, and End Results Program <https://seer.cancer.gov/statfacts/html/ovary.html>.
3. Fong P C, Boss D S, Yap T A, Tutt A, Wu P, Mergui-Roelvink M, Mortimer P, Swaisland H, Lau A, O'Connor M J, Ashworth A, Carmichael J, Kaye S B, Schellens J H, de Bono J S. Inhibition of poly(ADP-ribose) polymerase in tumors from BRCA mutation carriers. N Engl J Med 2009; 361(2):123-34 doi 10.1056/NEJMoa0900212.
4. Moore K, Colombo N, Scambia G, Kim B G, Oaknin A, Friedlander M, Lisyanskaya A, Floquet A, Leary A, Sonke G S, Gourley C, Banerjee S, Oza A, Gonzalez-Martin A, Aghajanian C, Bradley W, Mathews C, Liu J, Lowe E S, Bloomfield R, DiSilvestro P. Maintenance Olaparib in Patients with Newly Diagnosed Advanced Ovarian Cancer. N Engl J Med 2018; 379(26):2495-505 doi 10.1056/NEJMoa1810858.
5. Poveda A, Floquet A, Ledermann J A, Asher R, Penson R T, Oza A M, Korach J, Huzarski T, Pignata S, Friedlander M, Baldoni A, Park-Simon T-W, Sonke G S, Lisyanskaya A S, Kim J-H, Filho E A, Vergote I, Rowe P, Pujade-Lauraine E. Final overall survival (OS) results from SOL02/ENGOT-ov21: A phase III trial assessing maintenance olaparib in patients (pts) with platinum-sensitive, relapsed ovarian cancer and a BRCA mutation. Journal of Clinical Oncology 2020; 38(15_suppl):6002-doi 10.1200/JCO.2020.38.15_suppl.6002.
6. Robson M E, Tung N, Conte P, Im S A, Senkus E, Xu B, Masuda N, Delaloge S, Li W, Armstrong A, Wu W, Goessl C, Runswick S, Domchek S M. OlympiAD final overall survival and tolerability results: Olaparib versus chemotherapy treatment of physician's choice in patients with a germline BRCA mutation and HER2-negative metastatic breast cancer. Ann Oncol 2019; 30(4):558-66 doi 10.1093/annonc/mdz012.
7. Litton J K, Hurvitz S A, Mina L A, Rugo H S, Lee K H, Goncalves A, Diab S, Woodward N, Goodwin A, Yerushalmi R, Roche H, Im Y H, Eiermann W, Quek R G W, Usari T, Lanzalone S, Czibere A, Blum J L, Martin M, Ettl J. Talazoparib versus chemotherapy in patients with germline BRCA1/2-mutated HER2-negative advanced breast cancer: final overall survival results from the EMBRACA trial. Ann Oncol 2020; 31(11):1526-35 doi 10.1016/j.annonc.2020.08.2098.
8. Lord C J, Ashworth A. PARP inhibitors: Synthetic lethality in the clinic. Science 2017; 355(6330):1152-8 doi 10.1126/science.aam7344.
9. Ding L, Kim H J, Wang Q, Kearns M, Jiang T, Ohlson C E, Li B B, Xie S, Liu J F, Stover E H, Howitt B E, Bronson R T, Lazo S, Roberts T M, Freeman G J, Konstantinopoulos P A, Matulonis U A, Zhao J J. PARP Inhibition Elicits STING-Dependent Antitumor Immunity in Brca1-Deficient Ovarian Cancer. Cell Rep 2018; 25(11):2972-80 e5 doi 10.1016/j.celrep.2018.11.054.
10. Zou T, Zhou M, Gupta A, Zhuang P, Fishbein A R, Wei H Y, Capcha-Rodriguez D, Zhang Z, Cherniack A D, Meyerson M. XRN1 deletion induces PKR-dependent cell lethality in interferon-activated cancer cells. Cell Rep 2024; 43(2):113783 doi 10.1016/j.celrep.2024.113783.
11. Rehwinkel J, Mehdipour P. ADAR1: from basic mechanisms to inhibitors. Trends Cell Biol 2024 doi 10.1016/j.tcb.2024.06.006.
12. Gannon H S, Zou T, Kiessling M K, Gao G F, Cai D, Choi P S, Ivan A P, Buchumenski I, Berger A C, Goldstein J T, Cherniack A D, Vazquez F, Tsherniak A, Levanon E Y, Hahn W C, Meyerson M. Identification of ADAR1 adenosine deaminase dependency in a subset of cancer cells. Nat Commun 2018; 9(1):5450 doi 10.1038/s41467-018-07824-4.
13. Ishizuka J J, Manguso R T, Cheruiyot C K, Bi K, Panda A, Iracheta-Vellve A, Miller B C, Du P P, Yates K B, Dubrot J, Buchumenski I, Comstock D E, Brown F D, Ayer A, Kohnle I C, Pope H W, Zimmer M D, Sen D R, Lane-Reticker S K, Robitschek E J, Griffin G K, Collins N B, Long A H, Doench J G, Kozono D, Levanon E Y, Haining W N. Loss of ADAR1 in tumours overcomes resistance to immune checkpoint blockade. Nature 2019; 565(7737):43-8 doi 10.1038/s41586-018-0768-9.
14. Wang Q, Bergholz J S, Ding L, Lin Z, Kabraji S K, Hughes M E, He X, Xie S, Jiang T, Wang W, Zoeller J J, Kim H J, Roberts T M, Konstantinopoulos P A, Matulonis U A, Dillon D A, Winer E P, Lin N U, Zhao J J. STING agonism reprograms tumor-associated macrophages and overcomes resistance to PARP inhibition in BRCA1-deficient models of breast cancer. Nat Commun 2022; 13(1):3022 doi 10.1038/s41467-022-30568-1.
15. Bergholz J S, Wang Q, Wang Q, Ramseier M, Prakadan S, Wang W, Fang R, Kabraji S, Zhou Q, Gray G K, Abell-Hart K, Xie S, Guo X, Gu H, Von T, Jiang T, Tang S, Freeman G J, Kim H J, Shalek A K, Roberts T M, Zhao J J. PI3Kbeta controls immune evasion in PTEN-deficient breast tumours. Nature 2023; 617(7959):139-46 doi 10.1038/s41586-023-05940-w.
16. Robson M, Im S A, Senkus E, Xu B, Domchek S M, Masuda N, Delaloge S, Li W, Tung N, Armstrong A, Wu W, Goessl C, Runswick S, Conte P. Olaparib for Metastatic Breast Cancer in Patients with a Germline BRCA Mutation. N Engl J Med 2017; 377(6):523-33 doi 10.1056/NEJMoa1706450.
17. Kung C P, Cottrell K A, Ryu S, Bramel E R, Kladney R D, Bao E A, Freeman E C, Sabloak T, Maggi L, Jr., Weber J D. Evaluating the therapeutic potential of ADAR1 inhibition for triple-negative breast cancer. Oncogene 2021; 40(1):189-202 doi 10.1038/s41388-020-01515-5.
18. Chabanon R M, Muirhead G, Krastev D B, Adam J, Morel D, Garrido M, Lamb A, Henon C, Dorvault N, Rouanne M, Marlow R, Bajrami I, Cardenosa M L, Konde A, Besse B, Ashworth A, Pettitt S J, Haider S, Marabelle A, Tutt A N, Soria J C, Lord C J, Postel-Vinay S. PARP inhibition enhances tumor cell-intrinsic immunity in ERCC1-deficient non-small cell lung cancer. J Clin Invest 2019; 129(3):1211-28 doi 10.1172/JC1123319.
19. Pantelidou C, Sonzogni O, De Oliveria Taveira M, Mehta A K, Kothari A, Wang D, Visal T, Li M K, Pinto J, Castrillon J A, Cheney E M, Bouwman P, Jonkers J, Rottenberg S, Guerriero J L, Wulf G M, Shapiro G I. PARP Inhibitor Efficacy Depends on CD8(+) T-cell Recruitment via Intratumoral STING Pathway Activation in BRCA-Deficient Models of Triple-Negative Breast Cancer. Cancer Discov 2019; 9(6):722-37 doi 10.1158/2159-8290.CD-18-1218.
20. Platanias L C. Mechanisms of type-I- and type-II-interferon-mediated signalling. Nat Rev Immunol 2005; 5(5):375-86 doi 10.1038/nri1604.

What is claimed is:

1. A method of treating a cancer in a subject in need thereof, the method comprising:
administering to the subject a first pharmaceutical composition comprising an effective amount of an ADAR1 inhibitor and a pharmaceutically acceptable carrier; and
administering to the subject a second composition comprising an effective amount of a PARP inhibitor and a pharmaceutically acceptable carrier.

2. The method of claim 1, wherein the PARP inhibitor is selected from olaparib, talazoparib, and saruparib.

3. The method of claim 1, wherein the ADAR1 inhibitor comprises a gene silencing agent or a small molecule compound.

4. The method of claim 1, wherein the ADAR1 inhibitor comprises a gene silencing agent.

5. The method of claim 4, wherein the gene silencing agent is in a delivery vehicle.

6. The method of claim 5, wherein the delivery vehicle is a lentiviral vector or a nanocarrier.

7. The method of claim 4, wherein the gene silencing agent is selected from an siRNA, an shRNA, an miRNA, and an antisense oligonucleotide.

8. The method of claim 4, wherein the gene silencing agent is an siRNA.

9. The method of claim 8, wherein the siRNA comprises a sense strand comprising SEQ ID NO: 1, and an antisense strand comprising SEQ ID NO: 2.

10. The method of claim 4, wherein the gene silencing agent is an shRNA.

11. The method of claim 1, wherein the ADAR1 inhibitor is administered before the PARP inhibitor.

12. The method of claim 1, wherein the method further comprises administering to the subject an immunotherapy agent.

13. The method of claim 12, wherein the immunotherapy agent comprises an anti-PD-L1 antibody or an anti-PD-1 antibody.

14. The method of claim 1, wherein the subject has a breast cancer or an ovarian cancer.

15. The method of claim 1, wherein the subject has a cancer characterized by a loss of BRCA1 and p53.

16. The method of claim 1, wherein the subject has a cancer characterized by the loss of PTEN and p53.

17. The method of claim 1, wherein the subject is a human.
